(19) United States
(12) Patent Application Publication
(10) Pub. No.: US 2002/0074000 A1
(43) Pub. Date: Jun. 20, 2002

(54) Personal air quality unit

(76) Inventor: George Benda, Itasca, IL (US)

Correspondence Address: Clifford Kraft, 320 Robin Hill Dr., Naperville, IL, 60540, US

(21) Appl. No.: 10/012,251

(22) Filed: Nov. 5, 2001

Related U.S. Application Data
(60) Provisional application No. 60/246,438, filed on Nov. 7, 2000.

(51) Int. Cl. A62B 7/00; A62B 9/00; A61M 15/00; A61M 16/00; A62B 18/00; G08B 3/00; G08B 5/00; A62B 27/00; F16K 31/02; A62B 7/04; F16K 31/26

(52) U.S. Cl. 128/205.22; 128/204.29; 128/204.22; 128/202.22

(57) ABSTRACT

A personal pure air device where an air quality visualizer that can fit in a pocket like a pen samples ambient air quality for VOC's, poisonous gases, ozone, and possibly bacterial or viral content. The invention can contain a personal air bottle containing pure compressed air which can be dispensed through a regulator and tube to a nozzle to provide a plume of pure are around the user's nose and mouth providing a space of purified personal breathing air. The visualizer can be directly connected to the regulator to automatically start a flow of pure air when ambient air quality deteriorates, or the user can simply read the air visualizer and manually start the flow of pure air when desired through the use of valves.

This application is related to U.S. Provisional application No. 60/246,438 filed Nov. 7, 2000 and claims priority from it. Provisional application No. 60/246,438 is hereby incorporated by reference.

### BACKGROUND

1. Field of the Invention

This invention relates generally to the field of pure environmental air and more particularly to a personal pure air system.

2. Description of Related Art

Air is essential to life. Clean air is increasingly recognized as an important part of a healthy life. Concern about the quality of air has increased dramatically. Respiratory health in the general population is a public health concern reflected in the work of national and international environmental agencies such as the US EPA and the World Health Organization.

For the individual, concern about the quality of air can produce only alternative actions today; the individual may avoid places where there is a concern about air quality; they may use some form of prior art air cleaner, generally only for a larger space such as a room or house; or they may take radical steps, such as the use of various types of masks or respirators generally known in the art. The serious use of masks or respirators is generally reserved for known occupational hazards or for the handling of hazardous material by professionals. Typically, the individual can only guess by odors or irritation whether the quality of the air is poor or good. However, many dangerous contaminants are invisible and odorless. This results in uncertainty about the appropriateness of even the limited courses of action that are available.

What is badly needed is a personal air apparatus that both measures the quality of the air about an individual and also does something about it by creating a plume of purified air about the individual when and if the ambient air quality warrants it.

### SUMMARY OF THE INVENTION

The present invention relates to a personal air unit or apparatus that can be very small in physical size and can be portable. It can perform at least three functions: it can sense and indicate the quality of the air in the immediate vicinity of the user; it can provide a source of pure air on demand or when the sensor indicates unacceptable air quality; and it can dispense this clean air around the user's face to create a personal breathing zone of purified air.

The first function can be performed by part of the present invention that could be called an air visualizer. This can be a sensing and reporting device that collects a sample of air each time the cap is removed or at preselected intervals and illuminates a visual indicator to report whether selected air constituents are within certain parameters.

The second function can be performed by a pocket-sized container or metal or plastic that contains compressed, purified air. The bottle can be made of a lightweight material with sufficient structural strength to contain compressed air at a modest pressure. Purified air can be expelled from this bottle on demand from the user or when the air visualizer determines the necessity by sensing air that it determines is impure.

The third function can be performed by a device that can be called a personal air supplier. This device couples air from the bottled source to a pure breathing zone around the users head and face. The personal air supplier provides control over the rate of dispensing the bottled air and delivers a shaped stream of air through a micro-nozzle or other delivery means such as any other type of nozzle or airflow spreader. The pure air stream can be delivered directly into the breathing zone of the individual. Other additional functions can be performed by the present invention.

### BRIEF DESCRIPTION OF THE DRAWINGS

For a more complete understanding of this invention, reference should now be made to the embodiments illustrated in greater detail in the accompanying drawings.

FIGS. 1A and 1B show of an embodiment of an air visualizer.

FIG. 2 shows a sectional view of an embodiment of an air visualizer.

FIGS. 3A and 3B show an embodiment of an air container.

FIG. 4 shows a cross-section of an embodiment of an air dispensing control unit.

FIG. 5 shows an embodiment of the air dispensing control unit, dispensing tubing, and nozzle.

FIG. 6 shows a stick figure of a human wearing and using an embodiment of the present invention.

It should be noted that the above referenced figures are for illustrative purposes only and do not represent all embodiments of the present invention. One skilled in the art would recognize other embodiments and arrangements that are within the scope of the present invention.

### DETAILED DESCRIPTION

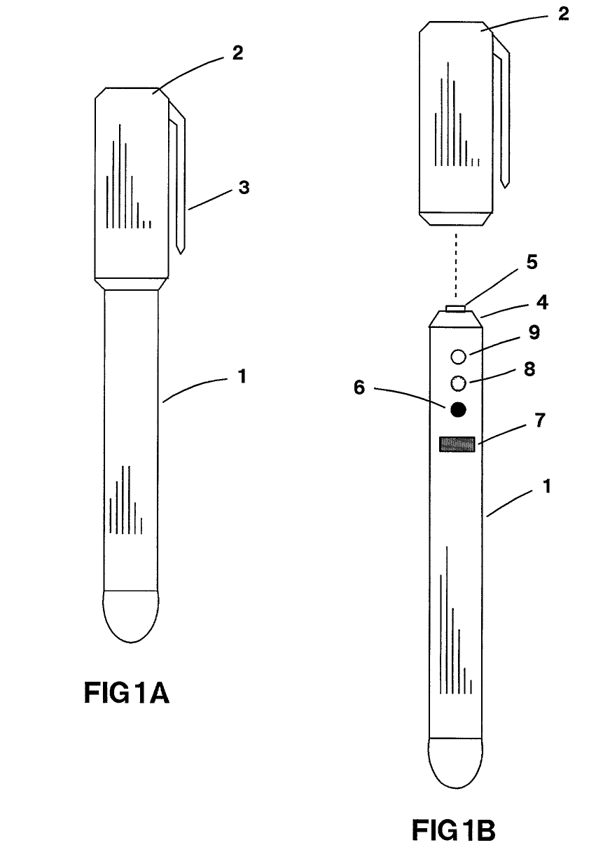

Turning to FIGS. 1A and 1B, an embodiment of an air visualizer is shown. An elongated tube 1 resembles a conventional pen with cap 2 and pocket clip 3. The cap 2 is removable as with a pen. An air inlet port 7 allows the device to sample ambient air at predetermined intervals. The preferred interval is around 5 to 10 seconds; however, many other sampling intervals are possible and are within the scope of the present invention. Another possible interval is to only sample once when the cap is removed.

The quality of the ambient air can be indicated by a series of miniature lights or LED devices 6, 8, 9 on the body of the elongated portion 1. A green light 6 could indicate that ambient air is clean and safe within predetermined parameters. An orange or yellow light 8 could indicate that ambient air is marginal with some undesirable compounds detected. A red light 9 could indicate that ambient air is not totally safe to breath. An optional audio alarm is also within the scope of the present invention. Such an audio alarm could activate when ambient air deteriorates to a certain unacceptable level. On the top of the lower body 5 can be a switch which, when depressed by the cap, could stop air sampling and shut the device off.

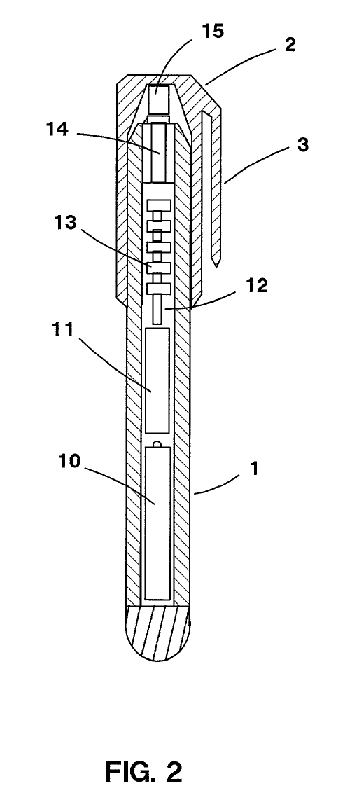

FIG. 2 shows a cross-section of an embodiment of an air visualizer. Contained within an elongated tube 1 can be a battery 10, an electronics package 11 and a series of sensors 13 that can sense ambient air for several different objectionable 10 compounds, materials, or biological entities. A switch 14, when depressed by a button 15 in the cap 2, can shut the unit off. The sensors 13 can sense for volatile organic compounds (VOC), poisonous gases such as carbon monoxide or hydrogen sulphide, ozone, NOx nitrous compounds, and many other objectionable compounds. Any sensor for any other compound is within the scope of the present invention. Biological sensors are also within the scope of the invention. These could sense for undesirable mold or fungi, bacteria or viruses. The sensors can be embedded on microchips that are mounted on disks 13 for use in the visualizer.

The electronics package 11 can power the sensors with power from the battery 10 and integrate results from the sensors in order to activate warning lights or alarms as previously described. The electronics package can contain a microcontroller or can be hardwired. The preferred method is to hardwire the electronic package to save space and power. However, in the future, smaller and less power-hungry processors will be developed. At that time, it may be preferable to use a processor or microcontroller. One job of the electronics package 11 is to read the output of the sensors 13 and determine the total amount of undesirable compounds in the air to come up with a total ambient air quality.

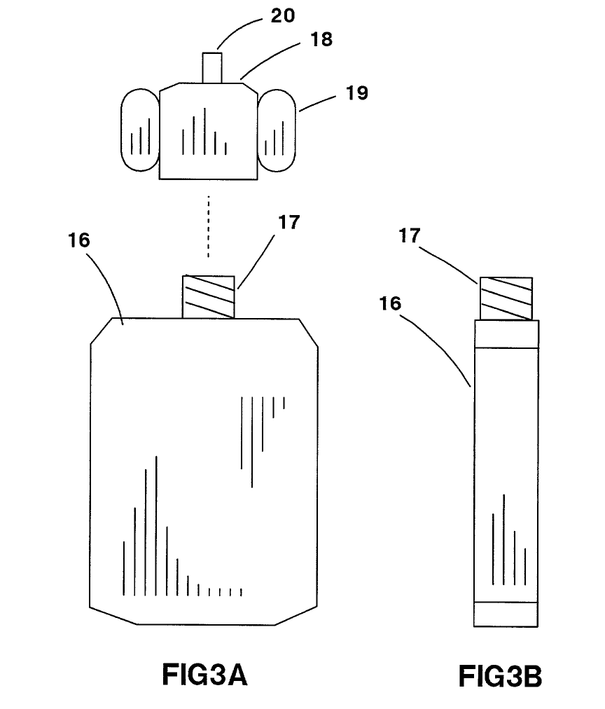

FIGS. 3A and 3B show an embodiment of an air bottle 16 that a person could carry on their person. This air bottle would hold pressurized fresh air at a moderate pressure. The pressure could be chosen to be within the safety limits of the bottle 16 and yet last long enough in use to be reasonable. At least an hour of continuous use would be preferred. The bottle 16 could be made from steel or hardened aluminum, or any material capable of being formed into a sealed pressure vessel.

The air bottle 16 can have a screw-on valve 17 on its top side or any other location. This coupling/valve 17 would allow a regulator 18 to fit over the bottle. The regulator 18 would lower and regulate the high pressure in the bottle 16, and present a constant lower pressure for distribution to a exit nipple 20. The regulator 18 could be equipped with at least one control valve 19 with which the user could increase or decrease the amount of personal air being supplied if the system is operated manually.

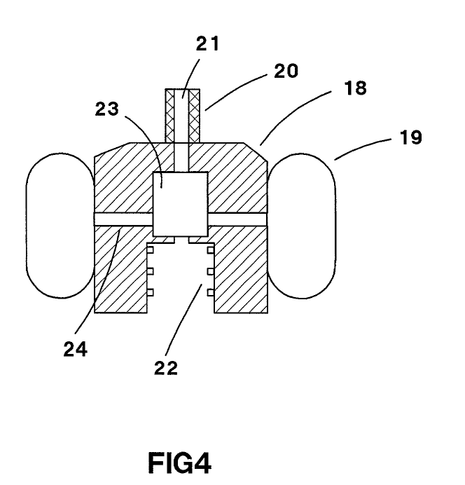

FIG. 4 shows a cross-section of an embodiment of a regulator 18. The regulator/valve unit screws over the personal air bottle 16 from FIG. 3A with a threaded cavity 22. A pin or other mechanism (not shown) would press open the valve of the air bottle allowing high pressure air to flow into the regulator. The actual regulator unit 23 is known in the art of converting variable high pressure to fixed low pressure flow. At least one valve 19 could control the amount of low pressure flow exiting the nipple 20 through its flow orifice 21.

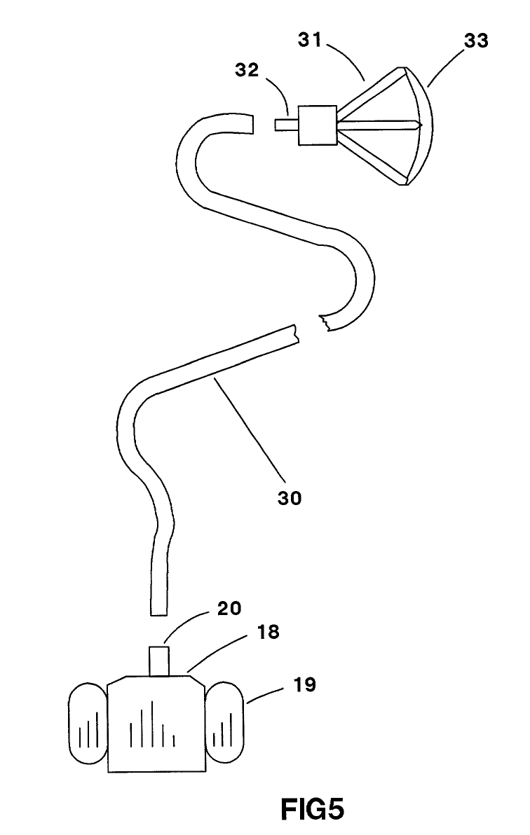

FIG. 5 shows a possible embodiment of the above described regulator 18 with valve 19 and nipple 20 where a plastic or other gas tube 30 would tightly fit over the nipple 20 and receive low pressure air from the regulator 18. A second end of the tube 30 would fit over a nipple 32 on a personal nozzle 31 so that a breathing space 33 of fresh air could be created in front of the nozzle 31.

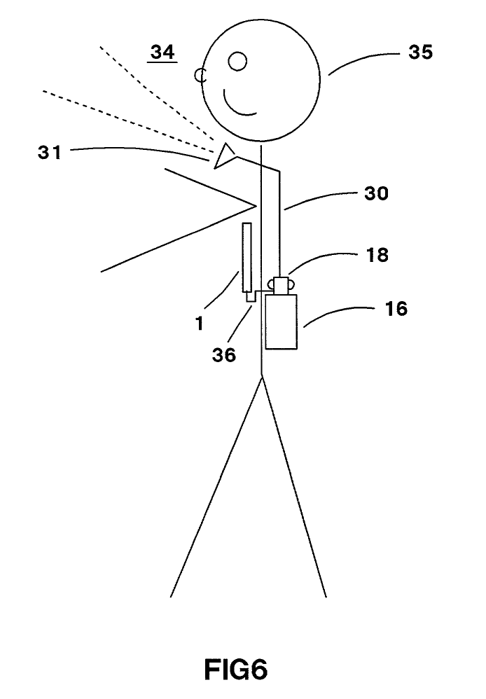

FIG. 6 shows a stick figure representation of a human 35 using an embodiment of the present invention. The personal air bottle 16 could be mounted on a belt, be held in a pocket, or be carried by any other means on the person. A regulator 18 would be screwed onto the air bottle 16 and the tube 30 could extend to the nozzle 31. A region or plume of pure breathable air 34 would be created around the user's mouth and nose so that the amount of inhaled contaminants would be greatly reduced. An air visualizer 1 can also be carried on the person to determine when pure air is needed.

A direct connection 36, electrical or otherwise, can be made between the air visualizer 1 and a valve/regulator unit 18 so that pure personal air is dispensed whenever ambient air quality deteriorates. Optionally, the air visualizer can be read by the person who then manually opens valves on the regulator 18 to start the flow of pure air.

It should be understood that the figures and embodiments shown herein are for illustration of the principles of the invention. Many other embodiments and arrangements would be understood by one skilled in the art and are within the scope of the present invention.

I claim:

1. A personal air supply system comprising:
an air visualizer means for determining ambient air quality;
a personal air bottle containing compressed purified air;
a nozzle for distributing purified air in a personal breathing space, said nozzle being coupled to said personal air bottle by a tube;
said personal air supply system dispensing purified air when said air visualizer determines reduced ambient air quality.

2. The personal air system of claim 1 further comprising direct connection between said air visualizer and said personal air bottle, said visualizer activating airflow when ambient air quality deteriorates.

3. The personal air system of claim 1 wherein said personal air bottle is metal.

4. The personal air system of claim 3 wherein said metal is steel.

5. The personal air system of claim 3 wherein said metal is aluminum.

6. The personal air system of claim 1 further comprising at least one light indicator of ambient air quality.

7. The personal air system of claim 6 further comprising an audio alarm indicator of ambient air quality.

8. The personal air system of claim 1 wherein said air visualizer contains a VOC sensor.

9. The personal air system of claim 1 wherein said air visualizer detects at least one poisonous gas.

10. A system for creating a pure air central breathing region around a user's nose and mouth when ambient air quality deteriorates, this system comprising a personal pure air bottle containing pressurized pure air, a regulator attached to the air bottle for producing a low pressure continuous flow of pure air, a tube for transporting the pure air to a nozzle, the nozzle being located near the user's nose and mouth.

11. The system of claim 10 further comprising an air visualizer with lights to inform the user when ambient air quality is deteriorating.

12. The system of claim 10 wherein the air visualizer contains at least on gas sensor.

13. The system of claim 12 wherein the gas sensor senses volatile organic compounds.

14. The system of claim 12 wherein the gas sensor senses at least one poisonous gas.

15. The system of claim 11 further comprising said air visualizer automatically starting said flow of pure air when said air visualizer detects deteriorating ambient air quality.

16. A method of providing a plume of pure air about a person's face when ambient air quality deteriorates comprising:
sampling air to continuously determine ambient air quality by using a system of gas sensors;
starting a flow of pure air from a personal air bottle carried on a person when ambient air quality deteriorates;
causing said flow of pure air to form a plume of pure air around a person's nose and mouth by using a tube and a nozzle;
stopping said flow of pure air when ambient air quality improves.

17. The method of claim 16 wherein said flow of pure air starts automatically when ambient air quality deteriorates.

18. The method of claim 16 wherein said flow of pure air is controlled manually and said ambient air quality is indicated with lights on an air visualizer.

19. The method of claim 18 further comprising an audio alarm on said air visualizer.

20. The method of claim 18 wherein said air visualizer detects at least one poisonous gas.
